(19) United States
(12) Patent Application Publication
(10) Pub. No.: US 2009/0269851 A1
(43) Pub. Date: Oct. 29, 2009

(54) USE OF DISK SURFACE FOR ELECTROPORATION OF ADHERENT CELLS

(75) Inventor: Charles W. Ragsdale, Concord, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: BIO-RAD LABORATORIES, INC. a corporation of the state of Delaware, Hercules, CA (US)

(21) Appl. No.: 12/425,141

(22) Filed: Apr. 16, 2009

Related U.S. Application Data
(60) Provisional application No. 61/047,637, filed on Apr. 24, 2008.

(51) Int. Cl. C12N 15/87; C12M 1/42

(52) U.S. Cl. 435/461; 435/285.2

(57) ABSTRACT

Adherent cells on the surface of a disk are transfected by electroporation between coaxial circular cylinders with electrodes on the opposing surfaces on either side of the annular space between the cylinders by placing a buffer solution containing the transfecting species in the annular space over the cell and applying an electric potential between the electrodes.

### CROSS-REFERENCE TO RELATED APPLICATION

This application claims the benefit of U.S. Provisional Patent Application No. 61/047,637, filed Apr. 24, 2008, the contents of which are incorporated herein by reference.

### BACKGROUND OF THE INVENTION

1. Field of the Invention

This invention lies in the field of transfection, the process by which exogenous molecular species are inserted into membranous structures by rendering the membrane permeable on a transient basis while the structures are in contact with a liquid solution of the species, thereby allowing the species to pass through the membrane, and doing so in such a manner that the structures resume their viability after the procedure is complete.

2. Description of the Prior Art

The introduction of exogenous species, including hydrophilic or membrane-impermeant species, into biological cells is of use in certain biologic and biochemical techniques. A high efficiency transfection is one in which the exogenous species has entered a high proportion of the cells or a population being treated and the viability of the cells has either been maintained throughout or restored after the procedure. Of the various transfection techniques, electroporation, which is the use of an electric field as the source of energy for the membrane permeabilization, has received the most attention. Transfection has been performed both on cells that are suspended in a buffer solution and on adherent cells, i.e, cells that are immobilized on a solid surface which is often the surface on which the cells have been grown. Achieving high efficiency is a continuing challenge in all forms of electroporation, but even more so in the electroporation of adherent cells. Disclosures of electroporation of adherent cells are found in the following published documents:

Jarvis et al., U.S. Pat. No. 6,897,069 B1, issued May 24, 2005

Lee et al., United States Patent Application Publication No. US 2007/0155016 A1, published Jul. 5, 2007

Vassanelli et al., United States Patent Application Publication No. US 2007/0115015 A1, published Jul. 5, 2007

Huang et al., United States Patent Application Publication No. US 2005/070510 A1, published Aug. 4, 2005

Acker, United States Patent Application Publication No. US 2004/0029240 A1, published Feb. 12, 2004

Zimmerman et al., United States Patent Application Publication No. US 2003/0148524 A1, published Aug. 7, 2003

Meyer, U.S. Pat. No. 6,261,815 B1, issued Jul. 17, 2001, issued Jul. 17, 2001

Korenstein et al., U.S. Pat. No. 5,964,726, issued Oct. 12, 1999

Casnig, U.S. Pat. No. 5,134,070, issued Jul. 28, 1992

Raptis, U.S. Pat. No. 6,001,617, issued Dec. 124, 1999

While the documents in the above list present a variety of approaches to improving the efficiency and uniformity of transfection, these qualities remain elusive and are a continuing goal.

### SUMMARY OF THE INVENTION

The cells to be transfected in accordance with this invention are grown or otherwise immobilized on the substantially flat surface of a circular disk in the area between the periphery of the disk and an inner circle within and concentric with the periphery. The cells thus occupy the surface of a flat ring. A cylinder with electrode material in the form of an electrode or a series of electrodes on its convex outward-facing surface and with a diameter that is equal to or less than the diameter of the inner circle serves as an inner electrode. A second, hollow cylinder with electrode material in the form of an electrode or series of electrodes on its concave inner surface encircles the periphery of the disk to serve as the outer electrode. The liquid solution of the exogenous species, referred to herein as the “transfecting species,” that will enter the cells is retained in a vessel that is large enough to receive both the disk and the electrodes with the disk surface immersed in the solution. The cylinder supporting the outer electrode(s) can itself serve as the vessel, with the outer electrodes on the inward-facing surface of the vessel. Whatever the configuration, the two electrodes (or series of electrodes) and the inner and outer electrodes are supported in the vessel in such a manner that the cells lie in a plane at the disk surface and the inner and outer electrodes both traverse the plane. The cylindrical surfaces on which the inner and outer electrodes reside form coaxial cylinders with a common cylinder axis, and the surface of the disk on which the cells reside is placed is transverse, and preferably perpendicular, to the axis. The electrodes when activated thus produce an electric field across the surface of the flat ring, exposing the cells on the surface to the field.

The inner electrode material resides on an outward-facing surface of a cylindrical support, and in certain embodiments of the invention, the disk and this cylindrical support with the inner electrode(s) on its surface are rigidly joined to form a unitary member. In other embodiments, the disk and the inner cylinder are separate components of the apparatus allowing cells to be grown on the disk surface without the presence of the inner cylinder. Regardless of whether the disk is integrated with the inner cylinder or separate, it is preferred that the disk be readily removable from the outer cylinder so that cells can be grown on the disk surface outside the apparatus. A disk that is removable from the outer cylinder offers flexibility and efficiency to the procedure by allowing cells to be grown on, or otherwise adhered to, one disk while cells on another disk are being subjected to electroporation. The disk can be placed in a vessel that allows the disk, or two or more disks, to be rotated while cells are being grown on its surface. The rotation agitates the surrounding growth medium and thereby improves access of the cells to the nutrients in the medium and to gases that promote cell growth. In certain embodiments of the invention, the disk can also be rotated inside the electroporation chamber (i.e., the outer cylinder) during electroporation.

In certain embodiments as well, the electrode material on the outer wall of the inner cylinder, i.e., the inner electrode, extends around the full circumference of the inner cylinder to form a continuous lining, while in other embodiments, this electrode material forms a patch or narrow strip affixed to the surface of the inner cylinder at a point on, or along a relatively short segment of, the circumference, or a series of patches or narrow strips distributed around the circumference, preferably with a uniform spacing. Similarly, the electrode material forming the outer electrode is either a continuous lining extending around the full circumference of the outer cylinder, a patch or strip, or a series of patches or strips to correspond to those on the inner cylinder. When at least one of the electrodes is not continuous around the full circumference of its respective cylinder, the disk in preferred embodiments is designed to be rotatable relative to both cylinders and hence to the electrodes, thereby allowing segments of the disk surface in succession to be placed in the space between the electrodes.

In certain embodiments of the invention, a small gap is maintained between the inner edge of the disk and the inner electrode, or between the outer edge of the disk and the outer electrode, or both. The gaps when present will avoid the formation of electric field anomalies at the edges of the disk. The inner and outer electrodes preferably extend both above and below the disk surface. In the method of use, a buffer solution containing the exogenous species is placed in the cylindrical receptacle formed by the outer cylinder with both electrodes in electrical contact with the solution and with the adherent cells immersed in the solution. The solution thus extends above the disk surface on which the cells reside, requiring no more than a shallow depth of liquid above the cells.

### BRIEF DESCRIPTION OF THE DRAWINGS

FIG. 1a is a side view of an integrated disk and inner cylinder for use in the present invention. FIG. 1b is a top view of the integrated disk and inner cylinder of FIG. 1a.

FIG. 2 is a cross section of a vessel with the integrated disk and inner cylinder of FIGS. 1a and 1b placed inside the vessel, and a solution of transfecting species covering the disk surface.

FIG. 3a is a top view of an alternative disk for sue in the present invention. FIG. 3b is a vertical cross section of the disk of FIG. 3a.

FIG. 4 is a cross section of a vessel with the disk of FIGS. 3a and 3b placed inside the vessel, together with an inner cylinder an a solution of transfecting species.

FIG. 5 is a diagram of a vessel for growing cells on several disks of the type shown in FIGS. 3a and 3b.

FIG. 6 is a top view of an inner and outer cylinder combination containing an alternative electrode arrangement, still within the scope of the invention.

FIG. 7 is a circuit diagram for the electrodes of FIG. 6.

### DETAILED DESCRIPTION OF THE INVENTION AND PREFERRED EMBODIMENTS

This invention is not restricted to disks of any particular size or size range. Preferably, however, the width of the ring-shaped area between the two cylindrical surfaces and on which the adherent cells reside is preferably small compared to the outer diameter of the disk so that the intensity of the electrical field created by the electrodes varies only minimally in the radial direction across the cell area when the electrodes fully extend around the circumferences of their respective cylinders. With a disk and electrodes of this shape, all cells adhering to the disk surface will experience a field intensity that is close to being uniform, or at least one that will not include a steep gradient in regions toward the center of the disk. With these considerations in mind, the ratio of the diameter of the inner edge to the diameter of the outer edge is preferably within the range of about 0.2 to about 0.95, more preferably the range of about 0.3 to about 0.9, and most preferably the range of about 0.5 to about 0.8. When a gap is present between either edge of the disk and the adjacent cylinder, the gap will provide room to allow the user to readily insert the disk, the inner cylinder, or an integrated disk and inner cylinder, into the outer cylinder and to remove it when desired. The gap is preferably small enough however to accommodate as wide a disk as possible and to thereby make maximal use of the distance between the electrodes for transfection of the cells. In most cases, a gap width within the range of about 30 microns to about 3 mm, more preferably the range of about 100 microns to about 1 mm, will be useful. The distance between electrodes facing each other across the width of the ring-shaped area on the disk can vary with the needs of the cells that are being transfected, although best results in most cases will be obtained with a distance in the range of about 0.3 cm to about 10 cm.

The disk surface can be fabricated of any material that is capable of serving as an immobilizing support for the cells. For biological cells, examples of suitable materials are glass, polycarbonate, polystyrene, polyvinyl, polyethylene, and polypropylene. Microporous membranes used in membrane-based cell culture can also be used. Examples are membranes of hydrophilic poly(tetrafluoroethylene), cellulose esters, polycarbonate, and polyethylene terephthalate. A membrane that is otherwise flexible can be maintained flat by placing the membrane over a rigid material such as a rigid screen or a glass or polymeric disk. Regardless of the surface composition, adherence of the cells to the surface can be achieved by conventional means, including the inherent adherence when the cells are grown on the surface, as well as adherence through immunological or affinity-type binding, electrostatic attraction, or covalent coupling.

The electrodes can be formed of electrode materials that are conventionally used in electroporation. The inner electrode can constitute the entire inner cylinder, which can either be of solid electrode material or hollow, and the outer electrode can likewise constitute the entire side wall of the chamber in which the ring, the inner electrode, and the buffer solution are retained. Alternatively, either or both of the inner and outer electrodes can be formed as surface layers, patches, or strips over electrically insulating materials. An electrically insulating cylindrical core, for example, can be plated on its outer surface with a metallic material to form the inner electrode, and an insulating shell can likewise be plated on its inner surface with a metallic material to form the outer electrode.

In use, the disk will be centered in the apparatus. For disks, and integrated disk-cylinder units, that are removable from the outer cylinder and that leave a gap between the outer edge of the disk and the outer cylinder, centering can be accomplished by any of various features. One example of a centering feature is a knob, or protrusion in general, at the underside of the disk, to mate with an indentation(s) in the floor of the chamber that is formed by the outer cylinder. When the disk is integrated with the inner cylinder, the protrusion can be centered on the underside of the inner cylinder. Alternatively, the protrusion(s) can extend upward from the floor of the chamber to mate with an indentation in the underside of the inner cylinder. When the disk is not joined to the inner cylinder, two or more protrusions or indentations can be arranged on the underside of the disk outside of the central opening of the disk. As a further alternative, a single circular ridge (or circular groove) mated with a complementary groove (or ridge) will serve the same function. Still further variations will be readily apparent, including those shown in the Figures and described below.

Centering of the inner cylinder itself, independently of the disk or with the disk attached, is likewise of value in certain embodiments of the invention. When the inner cylinder is integrated with the disk, the centering features described above will serve the purpose, and when the cylinder and the disk are separate, the cylinder can be supported from the top of the chamber with a centering feature. A further example of a centering feature that is integrated into the floor of the chamber and engages either the disk, the inner cylinder, or both is a magnet on one part and a facing insert of ferrous metal or other magnetically responsive material on the other. A magnet can thus be bonded or molded into the floor of the chamber, to attract a magnetically responsive metal bead, ball, or chip in the disk or the inner cylinder along the underside thereof, or vice versa. In certain embodiments, as will be seen below, further utility and benefits can be gained by including a magnet or magnetically active metal in a knob extending from the base of the disk or the inner cylinder when the disk is mounted to the inner cylinder. Still further examples of centering features are support brackets, a flange, or a shoulder extending from the inner cylinder, the outer cylinder, or both.

In the practice of this invention, the amount of the solution that is placed inside the chamber to contact the disk and cells adherent to the disk surface is not critical, but an advantage of this invention is that it allows one to use a very thin or shallow layer of the solution over the cells, and to thereby concentrate the electrical field over and around the cells. An amount of solution that is just enough to cover the cells can be used, and in general, the depth above the cells will preferably range from about 0.5 mm to about 5 mm.

Biological cells that can be transfected by the present invention include those that are grown on the disk surface and are naturally adherent thereto and those whose adherence is enhanced by cell-adhesive molecules that are either coupled to the cells or to the surface. All such cells are referred to herein as “adherent cells.” Examples of adherent cells are neuronal cells, neuronal stem cells, mesenchymal stem cells, pancreatic cells, skeletal muscle cells, cardiomyocytes, and liver or liver-derived cells such as primary hepatocytes, liver epithielial cells, HepG2 cells, and hepatocellular carcinoma-derived cells. Examples of the transfecting species to be inserted into the cells by the present invention are nucleic acids including DNA, RNA, plasmids, and genes and gene fragments, as well as proteins, pharmaceuticals, and enzyme cofactors. Further examples will be apparent to those skilled in the art.

While the features defining this invention are capable of implementation in a variety of constructions and procedures, the invention as a whole will be best understood by a detailed examination of certain specific embodiments such as those shown in the drawings.

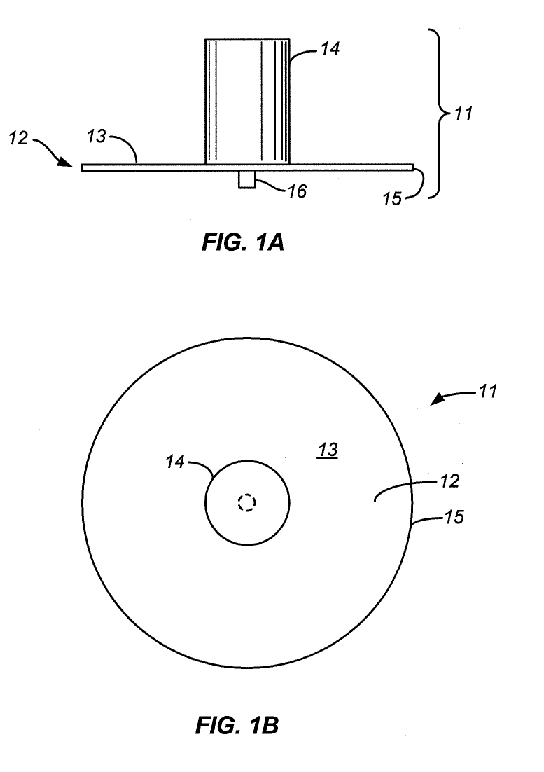

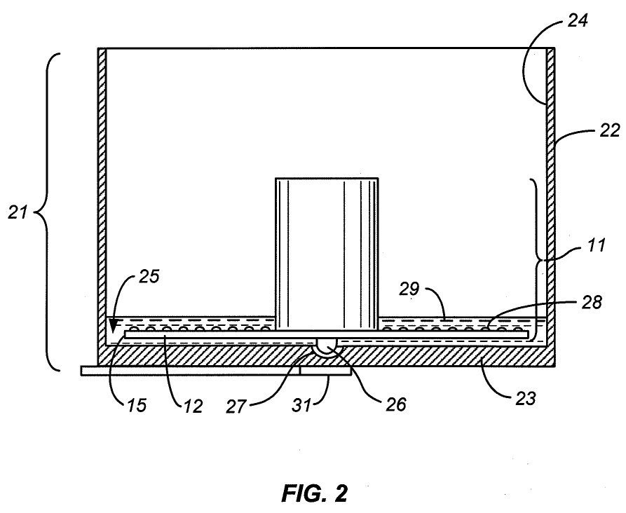

FIGS. 1a and 1b are two views of an integrated disk and inner cylinder 11, FIG. 1a showing the component from the side and FIG. 1b showing a top view. The disk 12 has a flat upper surface 13 bounded on the inside by the cylinder 14 and on the outside by a circular perimeter edge 15, the cylinder 14 and the perimeter edge 15 being concentric or coaxial. The cylinder 14 is either constructed of a continuous electrode material or of a core of non-electrode material with a layer of electrode material on its outer surface, in either case with an appropriate electrical connection between the electrode material and a power source (not shown). A knob 16 protrudes downward from the underside of the cylinder 14 at the center, and is shown as a small cylinder in these Figures but can also assume a variety of other shapes, including a hemisphere. The knob 16 is a centering feature and mates with an indentation of complementary shape in the floor of the chamber that is formed by the outer cylinder which is shown in FIG. 2. The centering feature can also include a magnet or a magnetically responsive metal, as discussed below. The knob 16 can also serve as the electrical connection that supplies power to the electrode, together with an electrical contact in the indentation to complete the circuit.

FIG. 2 shows the integrated disk and inner cylinder 11 of FIGS. 1a and 1b in position inside the chamber 21. The chamber 21, which is shown in cross section, is constructed of a cylindrical side wall 22 and a flat base 23, and in this particular example is open at the top. Electrode material constitutes either the entire side wall 22 of the cylinder or a layer on the inner surface 24 of the cylinder, with an appropriate electrical connection between the electrode material and the power source. The dimensions of the cylindrical wall 22 and the disk 12 are such that a small gap 25 exists between inner surface 24 of the side wall and the outer edge 15 of the disk. The central unit, i.e., the integrated disk and inner cylinder, can thus be easily inserted in the chamber and removed from the chamber, and electric field anomalies, at least at the outer edge of the disk, are avoided. In the construction shown in FIG. 2, the cylindrical knob 16 of FIGS. 1a and 1b is replaced by a hemispherical knob 26 that fits into a hemispherical indentation 27 in the flat base 23 of the chamber. Cells 28 into which the exogenous molecular species are to be inserted adhere to the upper surface of the ring 12, and the solution 29 containing the exogenous species covers the cells. For purposes of stabilization, the knob 26 contains magnetically responsive metal, and a magnet 31 is attached to the underside of the chamber by way of a removable support such as a sliding strip.

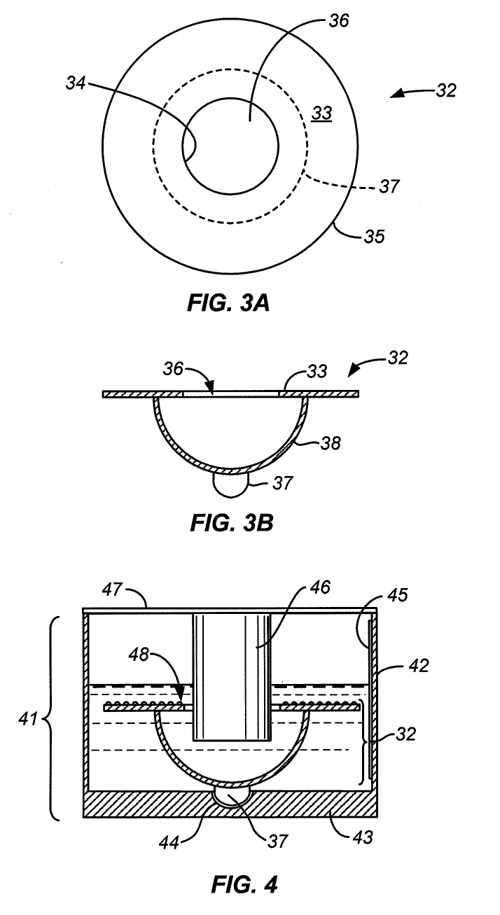

FIGS. 3a and 3b depict an alternative type of disk 32 that is separate from the inner cylinder. FIG. 3a is a top view of the disk and FIG. 3b is a side view in cross section. The surface 33 of this disk to which the cells adhere is of the same shape as, or a shape similar to, that of the surface of the disk in the preceding Figures, but the disk in FIGS. 3a and 3b has an exposed inner edge or rim 34 in addition to its exposed outer edge or periphery 35, the inner edge surrounding a central opening 36. A centering feature in the form of a hemispherical knob 37 protrudes from a bracket 38 extending downward from the disk. The bracket 38 is shown as a hollow hemisphere but can alternatively be a basket-type structure or a series of curved spokes.

FIG. 4 shows the disk 32 of FIGS. 3a and 3b inserted in a chamber 41. In a construction similar to the chamber 21 of FIG. 2, the chamber 41 in FIG. 4, which is likewise shown in cross section, has a cylindrical side wall 42 and a flat base 43, with an indentation 44 in the base that is complementary in contour to the knob 37 protruding from the disk bracket 38, and with an electrode layer 45 on the inner surface of the side wall. An inner cylindrical electrode 46 is mounted to a lid 47 that rests on the top of the cylindrical side wall 42 and extends into the chamber interior and through the central opening 36 of the disk 32. The central opening 36 is wide enough to leave a small gap 48 between the inner cylinder and the disk. The gap 48 permits easy insertion and removal of the inner cylinder 46, and reduces or eliminates electric field anomalies at the inner edge of the disk.

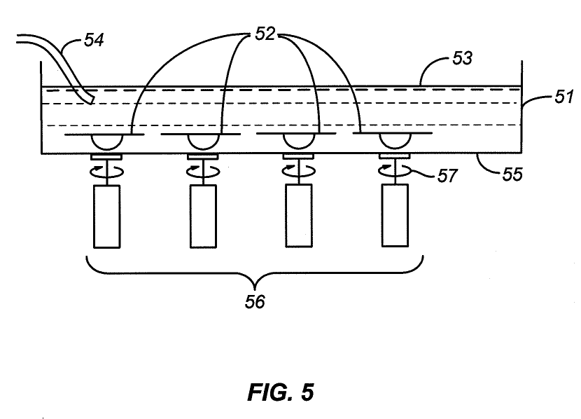

Each of FIGS. 1a, 1b, 2, 3a, 3b, and 4 depicts a knob attached to the disk. When the knob is magnetized or contains a magnetically responsive material, the disk can be rotated by a motorized rotating magnet, either in the chamber or in a separate vessel. Rotation of the disk in a separate vessel containing a growth medium offers a means of facilitating the growth of cells on the disk surface. An example of such a vessel is shown in FIG. 5. The vessel 51 in FIG. 5 accommodates several disks 52, or several units consisting of cylinders with integrated disks. The vessel 51 is filled with a liquid nutrient medium 53 and contains a source of nutrient gas 54 such as carbon dioxide. Beneath the floor 55 of the vessel are a series of rotary motors 56 each with a magnetic member 57. The rotating magnetic field produced by a rotating motor causes rotation of the disk whose protruding magnetic knob is in the magnetic field. The rotation enhances the access of the cells on the disk surface to the nutrients in the medium 53 and to the gas 54.

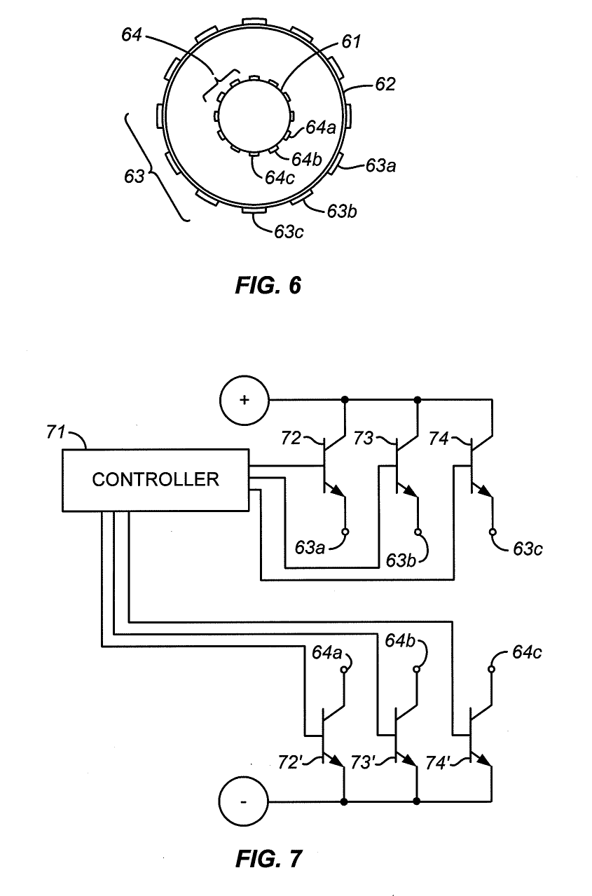

Each of the electrodes in the preceding Figures covers the entire circumference of a cylindrical surface, whether the surface be the outer surface of the inner cylinder or the inner surface of the chamber wall. FIG. 6 is an example of an apparatus in which series of discrete electrodes are used for both the inner and outer electrodes, the electrodes in each individual series being distributed around the full circumference of the cylinder on which the series is formed, and evenly spaced within each series. In a top view, FIG. 6 shows the inner cylinder 61 and the outer cylinder 62. A first series of strip electrodes 63 is secured to the outer surface of the outer cylinder 62 while a second series of strip electrodes 64 is secured to the outer surface of the inner cylinder 61. The two series are complementary, together forming pairs of electrodes facing each other across the space between the two cylinders. Although the disk is not shown, its construction and means of centering can be the same as those shown in the preceding Figures.

In the apparatus of FIG. 6, the electrodes can all be energized or pulsed simultaneously, or opposing pairs can be energized in succession around the disk. When pairs are individually energized, the resulting electric field will extend over cells occupying a wedge-shaped segment of the disk surface. A circuit for controlling the electrodes is shown in FIG. 7 in which circuit connections for only three electrode pairs 63a/64a, 63b/64b, 63c/64c, are shown for simplicity, the remaining connections being understood. A controller 71 controls a series of pairs of bipolar transistors 72/72′, 73/73′, 74/74′ corresponding with the individual electrode pairs in accordance with a selected protocol. The series of electrode pairs can also be replaced with a single electrode pair, i.e., one pair of the opposing strip electrodes shown in FIG. 6, and a rotating mechanism can be incorporated in the chamber to rotate the disk in increments to expose successive segments of the disk surface to the electric field pulses.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. An apparatus for transfection of adherent biological cells, said apparatus comprising:
a circular disk having a disk diameter and a substantially flat surface to which said cells adhere;
a vessel sized to retain a liquid solution and to receive said circular disk with said flat surface immersed in said liquid solution, said vessel having an inward-facing surface that forms a first circular cylinder having a diameter at least as great as said disk diameter, said inward-facing surface having electrode material thereon to form an outer electrode;
a cylindrical member having a diameter substantially less than that of said circular disk and an outward-facing surface that forms a second circular cylinder, said outward-facing surface having electrode material thereon to form an inner electrode;
means for supporting said circular disk and said cylindrical member within said vessel such that both said outer electrode and said inner electrode traverse a plane in which cells adherent on said flat surface reside; and
means for applying an electric potential between the outer and inner electrodes.

2. The apparatus of claim 1 further comprising means for centering said disk and said cylindrical member within said vessel.

3. The apparatus of claim 2 wherein said vessel has a floor, and said means for centering said disk comprises a protrusion extending from one side of said disk that mates with an indentation in said floor.

4. The apparatus of claim 2 wherein said vessel has a floor, and said means for centering said disk comprises a magnet in one of said disk and said floor and a magnetically responsive insert in the other of said disk and said floor.

5. The apparatus of claim 1 wherein said cylindrical member and said disk are rigidly joined to each other.

6. The apparatus of claim 1 wherein said disk and said cylindrical member are not connected to each other, and said disk has a central circular opening terminating in an inner rim large enough to receive said cylindrical member while leaving a gap around said cylindrical member and said rim.

7. The apparatus of claim 6 wherein said cylindrical member is affixed to a removable lid that covers said vessel.

8. The apparatus of claim 6 wherein said central opening has a diameter that is from about 0.3 times to about 0.9 times said disk diameter.

9. The apparatus of claim 1 wherein said outer electrode is a lining extending continuously around the circumference of said inward-facing surface.

10. The apparatus of claim 1 wherein said inner electrode is a lining extending continuously around the circumference of said outward-facing electrode.

11. The apparatus of claim 1 wherein said electrode material on said inward-facing surface of said vessel is a first series of discrete electrodes distributed around the circumference of said inward-facing surface, and said electrode material on said outward-facing surface of said cylindrical member is a second series of discrete electrodes distributed around the circumference of said outward-facing surface.

12. The apparatus of claim 1 further comprising means for rotating said disk.

13. The apparatus of claim 12 wherein said means for rotating said disk is a rotating magnet.

14. A process for the transfection of biological cells with a transfecting species, said process comprising:
immobilizing said cells on a surface of a substantially flat circular disk;
placing said cells so immobilized between electrodes on convex inner and concave outer cylindrical surfaces that form coaxial circular cylinders with a common cylinder axis, with said surface of said circular disk transverse to said axis; and
with said cells so placed and immersed in a liquid solution of said transfecting species, and with said electrodes in contact with said liquid solution, applying an electric potential between said electrodes on said inner and outer cylindrical surfaces sufficient to achieve transfection of said immobilized cells.

15. The process of claim 14 wherein said electrode residing on said convex inner cylindrical surface is a lining of electrode material extending continuously around the circumference of said convex inner cylindrical surface, and said electrode residing on said concave outer cylindrical surface is a lining of electrode material extending continuously around the circumference of said concave outer cylindrical surface.

16. The process of claim 14 wherein said electrodes residing on said concave outer cylindrical surface are a first series of discrete electrodes distributed around the circumference of said concave outer cylindrical surface, and said electrodes on said convex inner cylindrical surface are a second series of discrete electrodes distributed around the circumference of said convex inner cylindrical surface.

17. The process of claim 16 wherein said first and second series of discrete electrodes form a plurality of facing pairs of opposing electrodes, and said process further comprises energizing said facing pairs of opposing electrodes in sequence around said disk.

18. The process of claim 14 wherein said outer cylindrical surface is an inner surface of a vessel, and said process comprises immersing said disk in said liquid solution in said vessel.

19. The process of claim 14 wherein said outer cylindrical surface is an inner surface of a vessel, said convex inner cylindrical surface is the surface of a cylindrical insert, and cylindrical insert and said disk are joined to form a unitary member that fits inside said vessel.

20. The process of claim 14 further comprising rotating said flat disk around said cylinder axis.
